(19) United States
(12) Patent Application Publication
(10) Pub. No.: US 2024/0138980 A1
(43) Pub. Date: May 2, 2024

(54) Prosthetic Heart Valve Leaflet and Deployment Features

(71) Applicant: St. Jude Medical, Cardiology Division, Inc., St. Paul, MN (US)

(72) Inventors: Peter J. Ness, Minneapolis, MN (US); Tyler Govek, St. Louis Park, MN (US); Daniel J. Klima, Andover, MN (US); Kristopher Henry Vietmeier, Monticello, MN (US); Alexander Barrette, Maplewood, MN (US); William Minion, Vadnais Heights, MN (US); Janet Glenn, Forest Lake, MN (US)

(73) Assignee: St. Jude Medical, Cardiology Division, Inc., St. Paul, MN (US)

(21) Appl. No.: 18/487,006

(22) Filed: Oct. 13, 2023

Related U.S. Application Data
(60) Provisional application No. 63/381,878, filed on Nov. 1, 2022.

(51) Int. Cl. A61F 2/24

(52) U.S. Cl. CPC A61F 2/2418; A61F 2/2433; A61F 2250/001; A61M 2025/1059

(57) ABSTRACT

A prosthetic heart may include an expandable frame and prosthetic leaflets that each have a free edge, an attached edge having a contour, a first flat surface facing toward the frame, and a second flat surface facing away from the frame. A spacer fabric may be attached to each leaflet, the spacer fabric having a first surface, a second surface, and an inner matrix between the first and second surfaces. Each spacer fabric may follow the contour of the attached edge of a corresponding one of the prosthetic leaflets, and each spacer fabric may be wrapped around the contour of the attached edge of the corresponding one of the prosthetic leaflets such that the first surface of each spacer fabric contacts the first flat surface and the second flat surface of the corresponding prosthetic leaflet, each prosthetic leaflet being coupled to the frame via the corresponding spacer fabric

### CROSS-REFERENCE TO RELATED APPLICATIONS

This application claims the benefit of priority to U.S. Provisional Patent Application No. 63/381,878, filed Nov. 1, 2022, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves, including surgical heart valves and collapsible/expandable heart valves intended for transcatheter aortic valve replacement (“TAVR”) or transcatheter mitral valve replacement (“TMVR”), are well known in the patent literature. Surgical or mechanical heart valves may be sutured into a native annulus of a patient during an open-heart surgical procedure, for example. Collapsible/expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus.

Collapsible/expandable prosthetic heart valves typically take the form of a one-way valve structure (often referred to herein as a valve assembly) mounted to/within an expandable stent. In general, these collapsible/expandable heart valves include a self-expanding or balloon-expandable stent, often made of nitinol or another shape-memory metal or metal alloy (for self-expanding stents) or steel or cobalt chromium (for balloon-expandable stents). Existing collapsible/expandable TAVR devices have been known to use different configurations of stent layouts—including straight vertical struts connected by “V”s as illustrated in U.S. Pat. No. 8,454,685, or diamond-shaped cell layouts as illustrated in U.S. Pat. No. 9,326,856, both of which are hereby incorporated herein by reference. The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help retard leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

When self-expandable prosthetic heart valves are delivered into a patient to replace a malfunctioning native heart valve, the self-expandable prosthetic heart valve is almost always maintained in the collapsed condition within a capsule of the delivery device. While the capsule may ensure that the prosthetic heart valve does not self-expand prematurely, the overlying capsule (with or without the help of additional internal retaining features) helps ensure that the prosthetic heart valve does not come into contact with any tissue prematurely, as well as helping to make sure that the prosthetic heart valve stays in the desired position and orientation relative to the delivery device during delivery. However, balloon-expandable prosthetic heart valves are typically crimped onto the balloon of a delivery device without a separate capsule that overlies and/or protects the prosthetic heart valve.

Embodiments described herein may generally relate to features related to the connection of the prosthetic leaflets to the valve frame, features of inner and/or outer cuffs on the frame, and/or features for assisting with the deployment of the prosthetic valve into the native heart valve.

### BRIEF SUMMARY OF THE DISCLOSURE

According to one aspect of the disclosure, a prosthetic heart valve includes a collapsible and expandable frame and a plurality of prosthetic leaflets, each of the prosthetic leaflets having a free edge, an opposite attached edge having a contour, a first flat surface facing toward the frame, and a second flat surface facing away from the frame. A spacer fabric may be attached to each of the prosthetic leaflets, the spacer fabric having a first surface, a second surface, and an inner matrix between the first surface and the second surface. Each spacer fabric may follow the contour of the attached edge of a corresponding one of the prosthetic leaflets, and each spacer fabric may be wrapped around the contour of the attached edge of the corresponding one of the prosthetic leaflets such that the first surface of each spacer fabric contacts the first flat surface and the second flat surface of the corresponding prosthetic leaflet, each prosthetic leaflet being coupled to the frame via the corresponding spacer fabric. The first surface of each spacer fabric may be sutured to the corresponding prosthetic leaflet. The second surface of each spacer fabric may not be directly coupled to the corresponding prosthetic leaflet. The second surface of each spacer fabric may be sutured to the frame. The first surface of each spacer fabric may not be directly coupled to the frame. The first surface of each spacer fabric may be a finished surface, and the second surface of each spacer fabric may be a mat surface. The spacer fabric may be impregnated with collagen.

According to another aspect of the disclosure, a prosthetic heart valve may include a collapsible and expandable frame, a plurality of prosthetic leaflets, and an inner cuff coupled to a luminal surface of the frame. The inner cuff may be formed as a spacer fabric having a first surface that faces toward the frame, a second surface that faces away from the frame, and an inner matrix between the first surface and the second surface. The plurality of prosthetic leaflets may be coupled to the spacer fabric via sutures that pierce the second surface of the spacer fabric, but not the first surface of the spacer fabric. The sutures may directly couple the prosthetic leaflets to the second surface of the spacer fabric. Each of the prosthetic leaflets may have a free edge, an opposite attached edge having a contour, a first flat surface facing toward the frame, and a second flat surface facing away from the frame. A woven fabric may be sutured to each of the plurality of prosthetic leaflets so that each woven fabric follows the contour of the respective prosthetic leaflet. Each woven fabric may be directly sutured to the second surface of the spacer fabric, and each woven fabric may be directly sutured to the corresponding prosthetic leaflet, but the plurality of prosthetic leaflets may not be directly sutured to the spacer fabric. Each woven fabric may include a first fold in contact with the first flat surface of the corresponding prosthetic leaflet and a second fold in contact with the second flat surface of the corresponding prosthetic leaflet, the suturing of the woven fabric to the corresponding prosthetic leaflet passing through the first fold and the second fold.

According to a further aspect of the disclosure, a method of implanting a prosthetic heart valve includes advancing a delivery device to a native aortic valve of a patient while the prosthetic heart valve is crimped over a deflated balloon of the delivery device. While the prosthetic heart valve remains crimped over the deflated balloon, a position locator may be activated so that the position locator contacts a ventricular surface of the native aortic valve. While the position locator contacts the ventricular surface of the native aortic valve, the balloon may be inflated to expand the prosthetic heart valve into the native aortic valve. The position locator may be a braided mesh that overlies at least a leading edge of the prosthetic heart valve during the advancing. The position locator may include at least two wires that overlie at least a leading edge of the prosthetic heart valve during the advancing.

According to still another aspect of the disclosure, a method of implanting a prosthetic heart valve includes advancing a delivery device to a native aortic valve of a patient while the prosthetic heart valve is crimped over a balloon of the delivery device. While the prosthetic heart valve remains crimped over the balloon, a distal portion of the balloon positioned on a ventricular side of the native aortic valve may be inflated. Inflating the distal portion of the balloon may not expand the prosthetic heart valve into contact with the native aortic valve. After inflating the distal portion of the balloon, it may be confirmed that the inflated distal portion of the balloon is in contact with the ventricular side of the native aortic valve. After confirming, a second portion of the balloon may be inflated to expand the prosthetic heart valve into contact with the native aortic valve. The method may further include inflating a proximal portion of the balloon positioned on an aortic side of the native aortic valve before inflating the second portion of the balloon, so that the native aortic valve is sandwiched between the inflated proximal portion of the balloon and the inflated distal portion of the balloon.

According to still a further aspect of the disclosure, a method of implanting a prosthetic heart valve includes advancing a delivery device to a native aortic valve of a patient while the prosthetic heart valve is crimped over a deflated balloon of the delivery device and while an expandable member overlies at least a portion of the crimped prosthetic heart valve, an expansion limiter being coupled to the expandable member. While the prosthetic heart valve is aligned with the native aortic valve, the balloon may be inflated in a first phase of expansion to expand the prosthetic heart valve and the expandable member until the expansion limiter reaches a maximum diameter, the prosthetic heart valve not being in contact with the native aortic valve when the expansion limiter reaches the maximum diameter. After the first phase of expansion, the expandable member and the expansion limiter may be retracted so that the expandable member and the expansion member no longer overlie the prosthetic heart valve. The balloon may be inflated in a second phase of expansion to expand the prosthetic heart valve into contact with the native aortic valve. The expandable member may include two arms, and the expansion limiter may include two sutures or wires each coupled to the two arms.

According to yet another aspect of the disclosure, a method of implanting a prosthetic heart valve includes advancing a delivery device to a native aortic valve of a patient while the prosthetic heart valve is crimped over a deflated balloon of the delivery device. The balloon may begin to be inflated so that an outflow end of the prosthetic heart valve begins to expand before an inflow end of the prosthetic heart valve begins to expand. Inflation of the balloon may continue so that the inflow end of the prosthetic heart valve expands into contact with the native aortic valve.

According to yet a further aspect of the disclosure, a method of implanting a prosthetic heart valve includes advancing a delivery device to a native aortic valve of a patient while the prosthetic heart valve is crimped over a deflated balloon of the delivery device, the balloon including at least one fold at a proximal end portion of the balloon. The balloon may be inflated so that the prosthetic heart valve expands. The prosthetic heart valve may advance distally relative to the native aortic valve while the at least one fold at the proximal end portion of the balloon unfolds during the inflating. Inflation of the balloon may continue until the prosthetic heart valve expands into contact with the native valve annulus.

According to still another aspect of the disclosure, a method of implanting a prosthetic heart valve includes advancing a delivery device to a native aortic valve of a patient while the prosthetic heart valve is crimped over a deflated balloon of the delivery device, the delivery device including a radiopaque marker positioned proximal to an inflow end of the prosthetic heart valve. Under visualization, the radiopaque marker may be aligned with a desired target location of the native aortic valve so that the inflow end of the prosthetic heart valve is not aligned with the desired target location. After aligning the radiopaque marker, the balloon may be inflated to expand the prosthetic heart valve, the prosthetic heart valve axially foreshortening during the inflating. Inflation of the balloon may continue until the inflow end of the prosthetic heart valve contacts the desired target location of the native aortic valve.

### BRIEF DESCRIPTION OF THE DRAWINGS

FIG. 1A is a perspective view of a stent of a prosthetic heart valve according to an embodiment of the disclosure.

FIG. 1B is a schematic front view of a section of the stent of FIG. 1A.

FIG. 1C is a schematic front view of a section of a stent according to an alternate embodiment of the prosthetic heart valve of FIG. 1A.

FIGS. 1D-E are front views of the stent section of FIG. 1C in a collapsed and expanded state, respectively.

FIGS. 1F-G are side views of a portion of the stent according to the embodiment of FIG. 1C in a collapsed and expanded state, respectively.

FIG. 1H is a flattened view of the stent according to the embodiment of FIG. 1C, as if cut and rolled flat.

FIGS. 1I-J are front and side views, respectively, of a prosthetic heart valve including the stent of FIG. 1C.

FIG. 1K illustrates the view of FIG. 1H with an additional outer cuff provided on the stent.

FIG. 2A illustrates a prosthetic heart valve crimped over a balloon of a delivery device.

FIG. 2B is a schematic view of the balloon of FIG. 2A after having been inflated.

FIGS. 3A-B are views of a spacer fabric.

FIG. 4A is a schematic view of a prosthetic leaflet with an intermediate attachment member.

FIG. 4B is a schematic view of the prosthetic leaflet of FIG. 4A with through holes provided therethrough.

FIG. 4C is a schematic view of the prosthetic leaflet of FIG. 4A coupled to a frame of a prosthetic heart valve.

FIGS. 5A-C are schematic views of embodiments of woven fabrics for use as intermediate attachment members to prosthetic leaflets.

FIG. 6 is a schematic view of a delivery device for a prosthetic heart valve including position locators.

FIG. 7A is a schematic view of a delivery device for a prosthetic heart valve in a first stage of balloon expansion.

FIG. 7B is a schematic view of another embodiment of a delivery device for a prosthetic heart valve in a first stage of balloon expansion.

FIG. 8A is a schematic view of a delivery device for a prosthetic heart valve including expandable members and expansion limiters.

FIG. 8B1 is a schematic view of the delivery device of FIG. 8A in a first stage of expansion.

FIG. 8B2 is a schematic view of the expandable members and expansion limiters of FIG. 8A in a first stage of expansion.

FIG. 8C is a schematic view of the delivery device of FIG. 8A after partial retraction of the expandable members and expansion limiters.

FIG. 8D is a schematic view of the delivery device of FIG. 8B in a second stage of expansion.

FIG. 9A is a schematic view of a prosthetic heart valve before and after balloon expansion that illustrates axial foreshortening.

FIG. 9B is a schematic view of a prosthetic heart valve before and after a first stage of balloon expansion that illustrates axial foreshortening.

FIG. 9C is a schematic view of a prosthetic heart valve illustrating axial foreshortening and compensation for axial foreshortening.

FIG. 9D is a schematic view of a prosthetic heart valve crimped over a balloon with a folded portion.

FIG. 10 is a schematic view of a distal end of a delivery device for a balloon-expandable prosthetic heart valve having radiopaque markers thereon.

### DETAILED DESCRIPTION

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing the valve disclosed herein, however, it should be noted that the use of the valve is not limited to the intended position and orientation but may be deployed in any type of lumen or passageway. For example, although the prosthetic heart valve is described herein as a prosthetic aortic valve, the same or similar structures and features can be employed in other heart valves, such as the pulmonary valve, the mitral valve, or the tricuspid valve. Further, the term “proximal,” when used in connection with a delivery device or system, refers to a direction relatively close to the user of that device or system when being used as intended, while the term “distal” refers to a direction relatively far from the user of the device. In other words, the leading end of a delivery device or system is positioned distal to the trailing end of the delivery device or system, when being used as intended. As used herein, the terms “substantially,” “generally,” “approximately,” and “about” are intended to mean that slight deviations from absolute are included within the scope of the term so modified. As used herein, the stent may assume an “expanded state” and a “collapsed state,” which refer to the relative radial size of the stent.

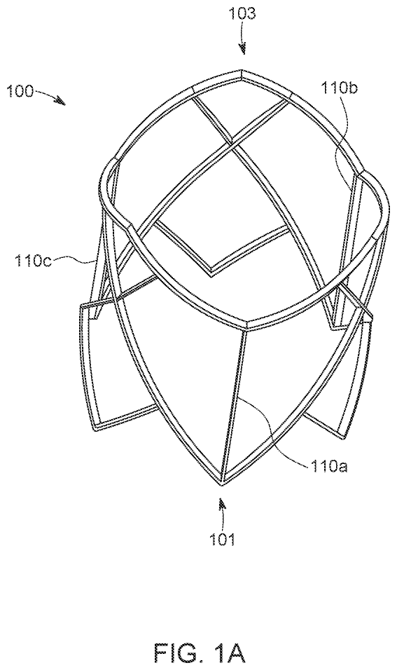

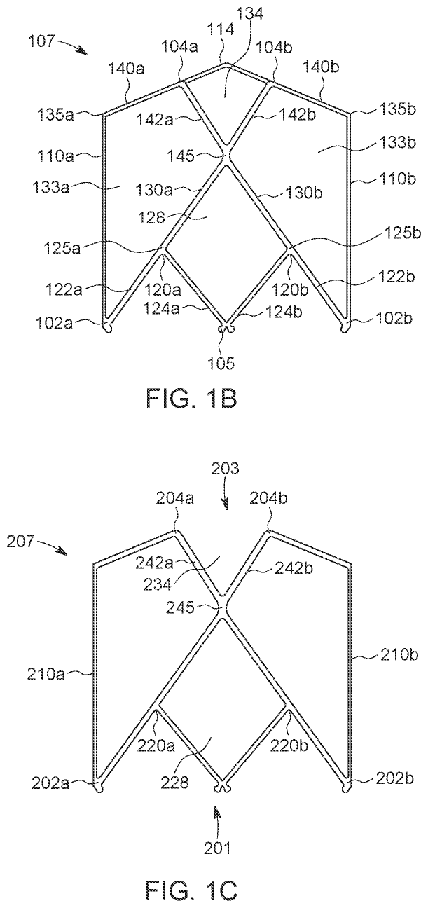

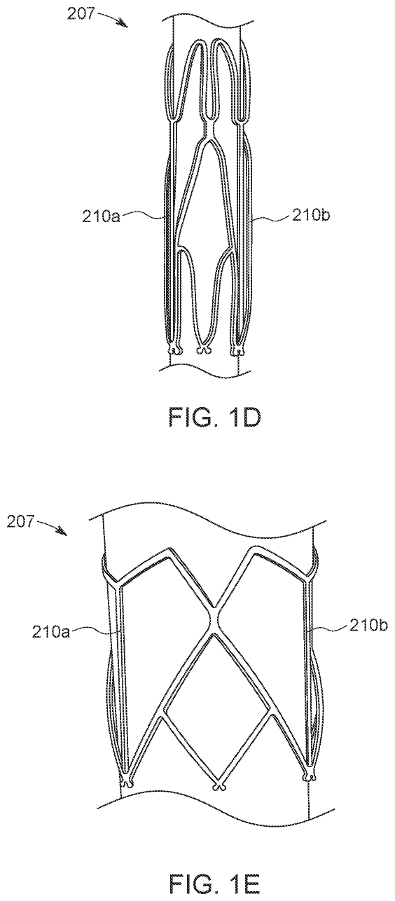

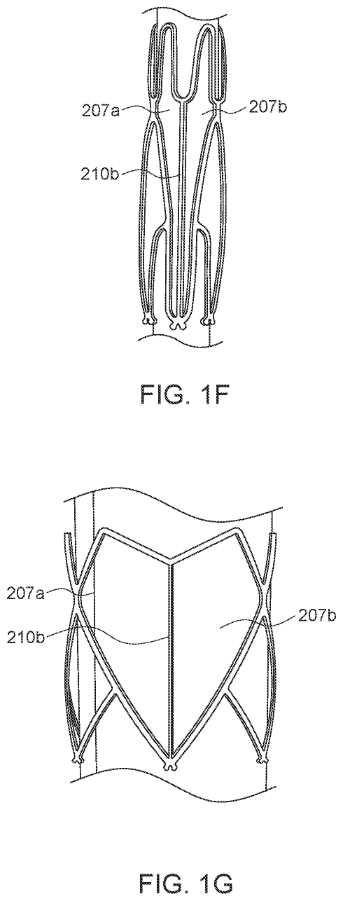

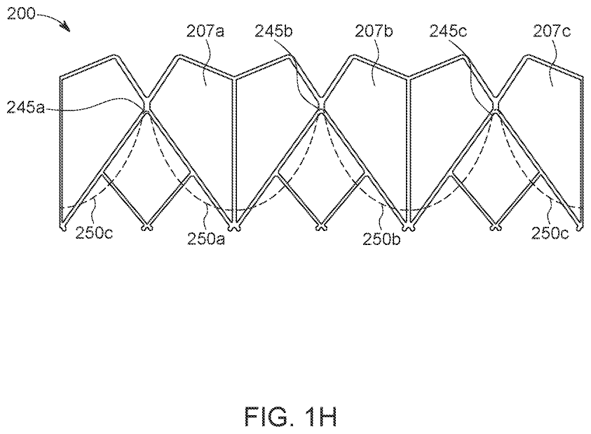

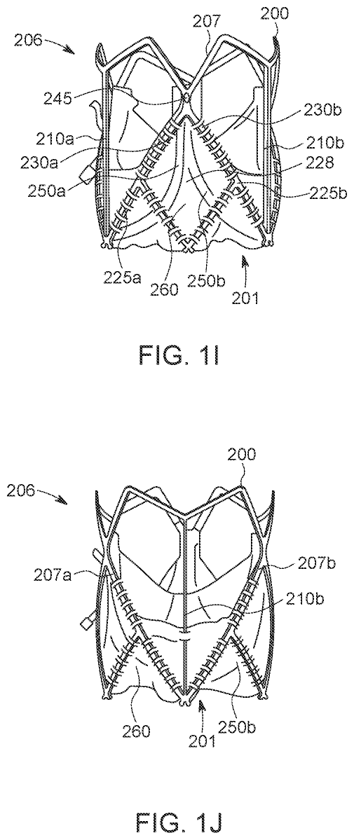

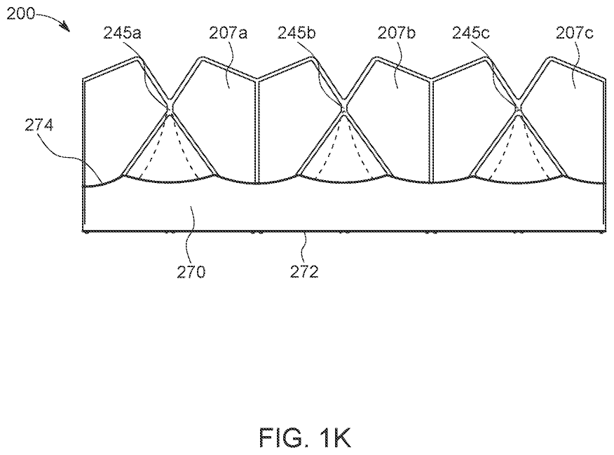

FIG. 1A illustrates a perspective view of a stent 100 of a prosthetic heart valve according to an embodiment of the disclosure. Stent 100 may include a frame extending in an axial direction between an inflow end 101 and an outflow end 103. Stent 100 includes three generally symmetric sections, wherein each section spans about 120 degrees around the circumference of stent 100. Stent 100 includes three vertical struts 110a, 110b, 110c, that extend in an axial direction substantially parallel to the direction of blood flow through the stent, which may also be referred to as a central longitudinal axis. Each vertical strut 110a, 110b, 110c may extend substantially the entire axial length between the inflow end 101 and the outflow end 103 of the stent 100 and may be disposed between and shared by two sections. In other words, each section is defined by the portion of stent 100 between two vertical struts. Thus, each vertical strut 110a, 110b, 110c is also separated by about 120 degrees around the circumference of stent 100. It should be understood that, if stent 100 is used in a prosthetic heart valve having three leaflets, the stent may include three sections as illustrated. However, in other embodiments, if the prosthetic heart valve has two leaflets, the stent may only include two of the sections.

FIG. 1B illustrates a schematic view of a stent section 107 of stent 100, which will be described herein in greater detail, and which is representative of all three sections. Stent section 107 depicted in FIG. 1B includes a first vertical strut 110a and a second vertical strut 110b. First vertical strut 110a extends axially between a first inflow node 102a and a first outer node 135a. Second vertical strut 110b extends axially between a second inflow node 102b and a second outer node 135b. As is illustrated, the vertical struts 110a, 110b may extend almost the entire axial length of stent 100. In some embodiments, stent 100 may be formed as an integral unit, for example by laser cutting the stent from a tube. The term “node” may refer to where two or more struts of the stent 100 meet one another. A pair of sequential inverted V's extends between inflow nodes 102a, 102b, which includes a first inflow inverted V 120a and a second inflow inverted V 120b coupled to each other at an inflow node 105. First inflow inverted V 120a comprises a first outer lower strut 122a extending between first inflow node 102a and a first central node 125a. First inflow inverted V 120a further comprises a first inner lower strut 124a extending between first central node 125a and inflow node 105. A second inflow inverted V 120b comprises a second inner lower strut 124b extending between inflow node 105 and a second central node 125b. Second inflow inverted V 120b further comprises a second outer lower strut 122b extending between second central node 125b and second inflow node 102b. Although described as inverted V's, these structures may also be described as half-cells, each half cell being a half-diamond cell with the open portion of the half-cell at the inflow end 101 of the stent 100.

Stent section 107 further includes a first central strut 130a extending between first central node 125a and an upper node 145. Stent section 107 also includes a second central strut 130b extending between second central node 125b and upper node 145. First central strut 130a, second central strut 130b, first inner lower strut 124a and second inner lower strut 124b form a diamond cell 128. Stent section 107 includes a first outer upper strut 140a extending between first outer node 135 and a first outflow node 104a. Stent section 107 further includes a second outer upper strut 140b extending between second outer node 135b and a second outflow node 104b. Stent section 107 includes a first inner upper strut 142a extending between first outflow node 104a and upper node 145. Stent section 107 further includes a second inner upper strut 142b extending between upper node 145 and second outflow node 104b. Stent section 107 includes an outflow inverted V 114 which extends between first and second outflow nodes 104a, 104b. First vertical strut 110a, first outer upper strut 140a, first inner upper strut 142a, first central strut 130a and first outer lower strut 122a form a first generally kite-shaped cell 133a. Second vertical strut 110b, second outer upper strut 140b, second inner upper strut 142b, second central strut 130b and second outer lower strut 122b form a second generally kite-shaped cell 133b. First and second kite-shaped cells 133a, 133b are symmetric and opposite each other on stent section 107. Although the term “kite-shaped,” is used above, it should be understood that such a shape is not limited to the exact geometric definition of kite-shaped. Outflow inverted V 114, first inner upper strut 142a and second inner upper strut 142b form upper cell 134. Upper cell 134 is generally kite-shaped and axially aligned with diamond cell 128 on stent section 107. It should be understood that, although designated as separate struts, the various struts described herein may be part of a single unitary structure as noted above. However, in other embodiments, stent 100 need not be formed as an integral structure and thus the struts may be different structures (or parts of different structures) that are coupled together.

FIG. 1C illustrates a schematic view of a stent section 207 according to an alternate embodiment of the disclosure. Unless otherwise stated, like reference numerals refer to like elements of above-described stent 100 but within the 200-series of numbers. Stent section 207 is substantially similar to stent section 107, including inflow nodes 202a, 202b, vertical struts 210a, 210b, first and second inflow inverted V's 220a, 220b and outflow nodes 204a, 204b. The structure of stent section 207 departs from that of stent section 107 in that it does not include an outflow inverted V. The purpose of an embodiment having such structure of stent section 207 shown in FIG. 1C is to reduce the required force to expand the outflow end 203 of the stent 200, compared to stent 100, to promote uniform expansion relative to the inflow end 201. Outflow nodes 204a, 204b are connected by a properly oriented V formed by first inner upper strut 242a, upper node 245 and second inner upper strut 242b. In other words, struts 242a, 242b may form a half diamond cell 234, with the open end of the half-cell oriented toward the outflow end 203. Half diamond cell 234 is axially aligned with diamond cell 228. Adding an outflow inverted V coupled between outflow nodes 204a, 204b contributes additional material that increases resistance to modifying the stent shape and requires additional force to expand the stent. The exclusion of material from outflow end 203 decreases resistance to expansion on outflow end 203, which may promote uniform expansion of inflow end 201 and outflow end 203. In other words, the inflow end 201 of stent 200 does not include continuous circumferential structure, but rather has mostly or entirely open half-cells with the open portion of the half-cells oriented toward the inflow end 201, whereas most of the outflow end 203 includes substantially continuous circumferential structure, via struts that correspond with struts 140a, 140b. All else being equal, a substantially continuous circumferential structure may require more force to expand compared to a similar but open structure. Thus, the inflow end 101 of stent 100 may require more force to radially expand compared to the outflow end 103. By omitting inverted V 114, resulting in stent 200, the force required to expand the outflow end 203 of stent 200 may be reduced to an amount closer to the inflow end 201.

FIG. 1D shows a front view of stent section 207 in a collapsed state and FIG. 1E shows a front view of stent section 207 in an expanded state. It should be understood that stent 200 in FIGS. 1D-E is illustrated with an opaque tube extending through the interior of the stent, purely for the purpose of helping illustrate the stent, and which may represent a balloon over which the stent section 207 is crimped. As described above, a stent comprises three symmetric sections, each section spanning about 120 degrees around the circumference of the stent. Stent section 207 illustrated in FIGS. 1D-E is defined by the region between vertical struts 210a, 210b. Stent section 207 is representative of all three sections of the stent. Stent section 207 has an arcuate structure such that when three sections are connected, they form one complete cylindrical shape. FIGS. 1F-G illustrate a portion of the stent from a side view. In other words, the view of stent 200 in FIGS. 1F-G is rotated about 60 degrees compared to the view of FIGS. 1D-E. The view of the stent depicted in FIGS. 1F-G is centered on vertical strut 210b showing approximately half of each of two adjacent stent sections 207a, 207b on each side of vertical strut 210b. Sections 207a, 207b surrounding vertical strut 210b are mirror images of each other. FIG. 1F shows stent sections 207a, 207b in a collapsed state whereas FIG. 1G shows stent sections 207a, 207b in an expanded state.

FIG. 1H illustrates a flattened view of stent 200 including three stent sections 207a, 207b, 207c, as if the stent has been cut longitudinally and laid flat on a table. As depicted, sections 207a, 207b, 207c are symmetric to each other and adjacent sections share a common vertical strut. As described above, stent 200 is shown in a flattened view, but each section 207a, 207b, 207c has an arcuate shape spanning 120 degrees to form a full cylinder. Further depicted in FIG. 1H are leaflets 250a, 250b, 250c coupled to stent 200. However, it should be understood that only the connection of leaflets 250a-c is illustrated in FIG. 1H. In other words, each leaflet 250a-c would typically include a free edge, with the free edges acting to coapt with one another to prevent retrograde flow of blood through the stent 200, and the free edges moving radially outward toward the interior surface of the stent to allow antegrade flow of blood through the stent. Those free edges are not illustrated in FIG. 1H. Rather, the attached edges of the leaflets 250a-c are illustrated in dashed lines in FIG. 1H. Although the attachment may be via any suitable modality, the attached edges may be preferably sutured to the stent 200 and/or to an intervening cuff or skirt between the stent and the leaflets 250a-c. Each of the three leaflets 250a, 250b, 250c, extends about 120 degrees around stent 200 from end to end and each leaflet includes a belly that may extend toward the radial center of stent 200 when the leaflets are coapted together. Each leaflet extends between the upper nodes of adjacent sections. First leaflet 250a extends from first upper node 245a of first stent section 207a to second upper node 245b of second stent section 207b. Second leaflet 250b extends from second upper node 245b to third upper node 245c of third stent section 207c. Third leaflet 250c extends from third upper node 245c to first upper node 245a. As such, each upper node includes a first end of a first leaflet and a second end of a second leaflet coupled thereto. In the illustrated embodiment, each end of each leaflet is coupled to its respective node by suture. However, any coupling means may be used to attach the leaflets to the stent. It is further contemplated that the stent may include any number of sections and/or leaflets. For example, the stent may include two sections, wherein each section extends 180 degrees around the circumference of the stent. Further, the stent may include two leaflets to mimic a bicuspid valve. Further, it should be noted that each leaflet may include tabs or other structures (not illustrated) at the junction between the free edges and attached edges of the leaflets, and each tab of each leaflet may be coupled to a tab of an adjacent leaflet to form commissures. In the illustrated embodiment, the leaflet commissures are illustrated attached to nodes where struts intersect. However, in other embodiments, the stent 200 may include commissure attachment features built into the stent to facilitate such attachment. For example, commissures attachment features may be formed into the stent 200 at nodes 245a-c, with the commissure attachment features including one or more apertures to facilitate suturing the leaflet commissures to the stent. Further, leaflets 250a-c may be formed of a biological material, such as animal pericardium, or may otherwise be formed of synthetic materials, such as plastics, fabrics, and/or polymers, including ultra-high molecular weight polyethylene (UHMWPE).

FIGS. 1I-J illustrate prosthetic heart valve 206, which includes stent 200, a cuff 260 coupled to stent 200 (for example via sutures) and leaflets 250a, 250b, 250c attached to stent 200 and/or cuff 260 (for example via sutures). Prosthetic heart valve 206 is intended for use in replacing an aortic valve, although the same or similar structures may be used in a prosthetic valve for replacing other heart valves. Cuff 260 is disposed on a luminal or interior surface of stent 200, although the cuff could be disposed alternately or additionally on an abluminal or exterior surface of the stent. The cuff 260 may include an inflow end disposed substantially along inflow end 201 of stent 200. FIG. 1I shows a front view of valve 206 showing one stent portion 207 between vertical struts 210a, 210b including cuff 260 and an outline of two leaflets 250a, 250b sutured to cuff 260. Different methods of suturing leaflets to the cuff as well as the leaflets and/or cuff to the stent may be used, many of which are described in U.S. Pat. No. 9,326,856 which is hereby incorporated by reference. In the illustrated embodiment, the upper (or outflow) edge of cuff 260 is sutured to first central node 225a, upper node 245 and second central node 225b, extending along first central strut 230a and second central strut 230b. The upper (or outflow) edge of cuff 260 continues extending approximately between the second central node of one section and the first central node of an adjacent section. Cuff 260 extends between upper node 245 and inflow end 201. Thus, cuff 260 covers the cells of stent portion 207 formed by the struts between upper node 245 and inflow end 201, including diamond cell 228. FIG. 1J illustrates a side view of stent 200 including cuff 260 and an outline of leaflet 250b. In other words, the view of valve 206 in FIG. 1J is rotated about 60 degrees compared to the view of FIG. 1I. The view depicted in FIG. 1J is centered on vertical strut 210b showing approximately half of each of two adjacent stent sections 207a, 207b on each side of vertical strut 210b. Sections 207a, 207b surrounding vertical strut 210b are mirror images of each other. As described above, the cuff may be disposed on the stent's interior or luminal surface, its exterior or abluminal surface, and/or on both surfaces. A cuff ensures that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular leakage or “PV” leakage). In the embodiment illustrated in FIGS. 1I-J, the cuff 260 only covers about half of the stent 200, leaving about half of the stent uncovered by the cuff. With this configuration, less cuff material is required compared to a cuff that covers more or all of the stent 200. Less cuff material may allow for the prosthetic heart valve 206 to crimp down to a smaller profile when collapsed. It is contemplated that the cuff may cover any amount of surface area of the cylinder formed by the stent. For example, the upper edge of the cuff may extend straight around the circumference of any cross section of the cylinder formed by the stent. Cuff 260 may be formed of any suitable material, including a biological material such as animal pericardium, or a synthetic material such as UHMWPE.

As noted above, FIGS. 1I-J illustrate a cuff 260 positioned on an interior of the stent 200. An example of an additional outer cuff 270 is illustrated in FIG. 1K. It should be understood that outer cuff 270 may take other shapes than that shown in FIG. 1K. The outer cuff 270 shown in FIG. 1K may be included without an inner cuff 260, but preferably is provided in addition to an inner cuff 260. The outer cuff 270 may be formed integrally with the inner cuff 260 and folded over (e.g. wrapped around) the inflow edge of the stent, or may be provided as a member that is separate from inner cuff 260. Outer cuff 270 may be formed of any of the materials described herein in connection with inner cuff 260. In the illustrated embodiment, outer cuff 270 includes an inflow edge 272 and an outflow edge 274. If the inner cuff 260 and outer cuff 270 are formed separately, the inflow edge 272 may be coupled to an inflow end of the stent 200 and/or an inflow edge of the inner cuff 260, for example via suturing, ultrasonic welding, or any other suitable attachment modality. The coupling between the inflow edge 272 of the outer cuff 270 and the stent 200 and/or inner cuff 260 preferably results in a seal between the inner cuff 260 and outer cuff 270 at the inflow end of the prosthetic heart valve so that any retrograde blood that flows into the space between the inner cuff 260 and outer cuff 270 is unable to pass beyond the inflow edges of the inner cuff 260 and outer cuff 270. The outflow edge 274 may be coupled at selected locations around the circumference of the stent 200 to struts of the stent 200 and/or to the inner cuff 260, for example via sutures. With this configuration, an opening may be formed between the inner cuff 260 and outer cuff 270 circumferentially between adjacent connection points, so that retrograde blood flow will tend to flow into the space between the inner cuff 260 and outer cuff 270 via the openings, without being able to continue passing beyond the inflow edges of the cuffs. As blood flows into the space between the inner cuff 260 and outer cuff 270, the outer cuff 270 may billow outwardly, creating even better sealing between the outer cuff 270 and the native valve annulus against which the outer cuff 270 presses. The outer cuff 270 may be provided as a continuous cylindrical member, or a strip that is wrapped around the outer circumference of the stent 200, with side edges, which may be parallel or non-parallel to a center longitudinal axis of the prosthetic heart valve, attached to each other so that the outer cuff 270 wraps around the entire circumference of the stent 200.

The stent may be formed from biocompatible materials, including metals and metal alloys such as cobalt chrome (or cobalt chromium) or stainless steel, although in some embodiments the stent may be formed of a shape memory material such as nitinol or the like. The stent is thus configured to collapse upon being crimped to a smaller diameter and/or expand upon being forced open, for example via a balloon within the stent expanding, and the stent will substantially maintain the shape to which it is modified when at rest. The stent may be crimped to collapse in a radial direction and lengthen (to some degree) in the axial direction, reducing its profile at any given cross-section. The stent may also be expanded in the radial direction and foreshortened (to some degree) in the axial direction.

The prosthetic heart valve may be delivered via any suitable transvascular route, for example including transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing the valve is inserted through the femoral artery and threaded against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstance, under an overlying sheath). Upon arrival at or adjacent the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

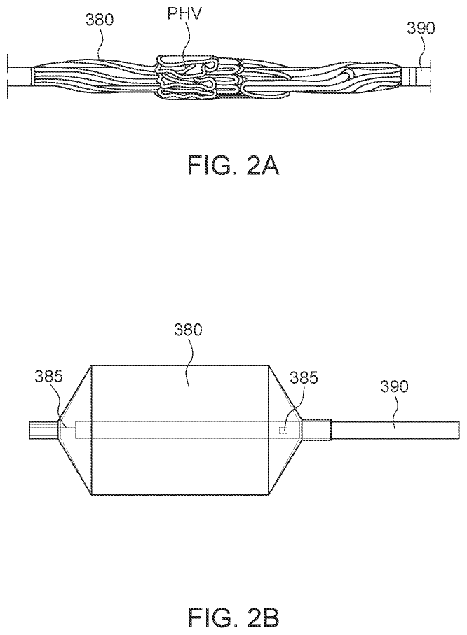

Referring to FIG. 2A, an example of a prosthetic heart valve PHV, which may include a stent similar to stents 100 or 200, is shown crimped over a balloon 380 of a balloon catheter 390 while the balloon 380 is in a deflated condition. It should be understood that other components of the delivery device, such as a handle used for steering and/or deployment, as well as a syringe for inflating the balloon 380, are omitted from FIGS. 2A-B. The prosthetic heart valve PHV may be delivered intravascularly, for example through the femoral artery, around the aortic arch, and into the native aortic valve annulus, while in the crimped condition shown in FIG. 2A. Once the desired position is obtained, fluid may be pushed through the balloon catheter 390 to inflate the balloon 380, as shown in FIG. 2B. FIG. 2B omits the prosthetic heart valve PHV, but it should be understood that, as the balloon 380 inflates, it forces the prosthetic heart valve PHV to expand into the native aortic valve annulus (although it should be understood that other heart valves may be replaced using the concepts described herein). In the illustrated example, fluid flows from a syringe (not shown) into the balloon 380 through a lumen within balloon catheter 390 and into one or more ports 385 located internal to the balloon 380. In the particular illustrated example of FIG. 2B, a first port 385 may be one or more apertures in a side wall of the balloon catheter 390, and a second port 385 may be the distal open end of the balloon catheter 390, which may terminate within the interior space of the balloon 380.

During normal operation of a prosthetic heart valve, the prosthetic leaflets open and close cyclically as the chambers of the heart contract and relax. For example, when the left ventricle relaxes and the left atrium contracts, the mitral valve opens and the aortic valve closes. For a prosthetic aortic valve, as the left ventricle relaxes, the prosthetic leaflets coapt to prevent blood from flowing in the retrograde direction from the aorta back into the left ventricle. As the prosthetic leaflets open and close, and particularly when they close, the prosthetic leaflets can encounter stress as the prosthetic leaflets resist the pressure gradient across the closed valve assembly. This stress may largely act at the point(s) where the prosthetic leaflets are affixed to the frame (or an intermediary component). Because prosthetic heart valves may need to last years, decades, or more, it may be important to minimize the amount of stress experienced by the prosthetic leaflets during normal operation to reduce the amount of wear and tear on the prosthetic leaflets, since such wear and tear may reduce the longevity of the prosthetic leaflets. One way to reduce stress on the prosthetic leaflets is to allow for deflection of the structure to which the prosthetic leaflets are attached. For example, if prosthetic leaflets are directly sutured to a commissure attachment feature of a frame, allowing the frame to deflect slightly (e.g., about 1 mm) as the prosthetic leaflets close may help reduce the stresses on the prosthetic leaflets as they coapt.

Self-expanding prosthetic heart valves typically include frames that are formed of nitinol or a similar material. Nitinol is a generally flexible material and when prosthetic leaflets are directly sutured to a nitinol frame, the flexibility of the nitinol may inherently provide for deflection at the point of connection to the prosthetic leaflets, resulting in smaller stresses on the prosthetic leaflets at their connection locations. However, balloon-expandable prosthetic heart valves may include a frame formed of cobalt-chromium (or cobalt chromium with nickel), which is typically a significantly stiffer or more rigid material than nitinol. Because cobalt chromium is stiffer, it may have less natural deflection compared to nitinol stents. As a result, for two otherwise identical prosthetic heart valves, one with a cobalt-chromium frame will typically result in greater stress on the prosthetic leaflets at their connection point to the frame compared to a nitinol frame. Thus, especially for prosthetic heart valves with stiffer frames, it may be desirable to provide alternate mechanisms for creating deflection or otherwise reducing stress on the prosthetic leaflets as they close.

According to one aspect of the disclosure, an intermediate connection member may be used to avoid coupling the prosthetic leaflets directly to the frame of the prosthetic heart valve. The frame of the prosthetic heart valve may be similar or identical to frame 100 or frame 200 described above, but it should be understood that the intermediate connection member may be used with various other designs of prosthetic heart valve frames. As should be understood, the intermediate connection member described below may be most useful with prosthetic heart valves having frames that are relatively stiff or rigid, such as frames formed of cobalt-chromium, but the intermediate connection members may be used with prosthetic heart valves having frames formed of other materials, including nitinol.

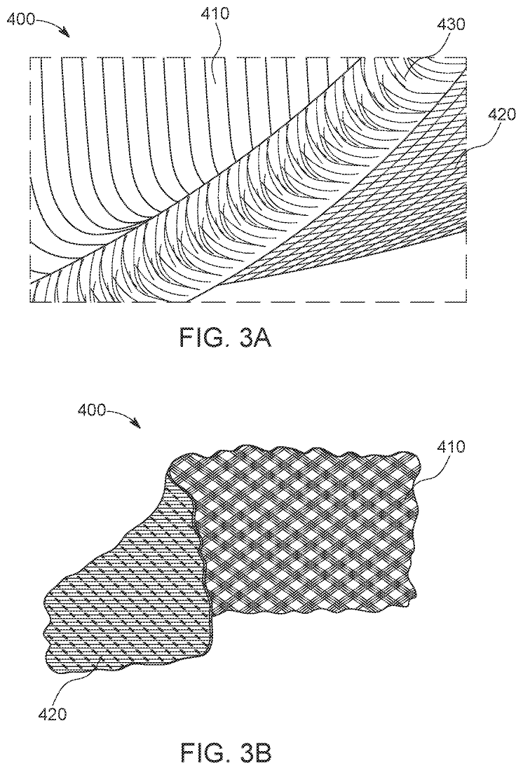

FIGS. 3A-B illustrate an exemplary material that may be used as an intermediate connection member 400. The intermediate connection member 400 shown in FIGS. 3A-B may have a structure that is referred to as a spacer fabric and may include two sides and an inner matrix. In some embodiments, the spacer fabric 400 may include a finished surface or side 410, a mat surface or side 420, and an inner matrix 430 between the two sides 410, 420 (best illustrated in FIG. 3A). However, it should be understood that the spacer fabric 400 shown in FIGS. 3A-B is only one example of a material that may be used for the intermediate connection member 400 described below. In one example, the finished side 410 may be an open cell knit (as shown in FIG. 3B), or a tighter knit (similar to the mat side 420 described below). The loft (e.g., the height of the knit) of the finished side 410 may be adjusted as desired, as may be the number of loops in the knit. The mat side 420 may be more tightly woven than the finished side 410, and as described below, may be configured to face the interior of the prosthetic heart valve if being used as an inner cuff. The thread used to knit the spacer fabric 400 may be monofilament or multi-filament strands, and in some embodiments may be Nylon, PET, PTFE, TPU or UHMWPE. While knitting the spacer fabric 400 shown in FIGS. 3A-B, the material characteristics of each side of the knit can be controlled, such as the knit density, structure (e.g., hole size/shape/pattern). Similarly, the fiber that is used on each side of the spacer fabric 400 may be controlled. Depending on the stresses that each side of the spacer fabric 400 is expected to be exposed to, as well as the desired attachment processes, the above-noted characteristics may be manipulated to achieve a desired final result. In some examples of the spacer fabric 400 described above, the inner matrix 430 includes fibers normal (or substantially normal) to the plane of the mat side 420 and/or finished side 410. The properties of the inner matrix 430 (e.g., density, fiber height, fiber density, mono/multi-filament, etc.) may be controlled during production to get desired characteristics such as density, porosity, and/or compressibility/thickness. Any standard polymer used for weaving/knitting may be used to form the spacer fabric 400, including e.g., PET, PTFE, UHMWPE, etc. Multiple materials may also be used for the same final design for the spacer fabric 400.

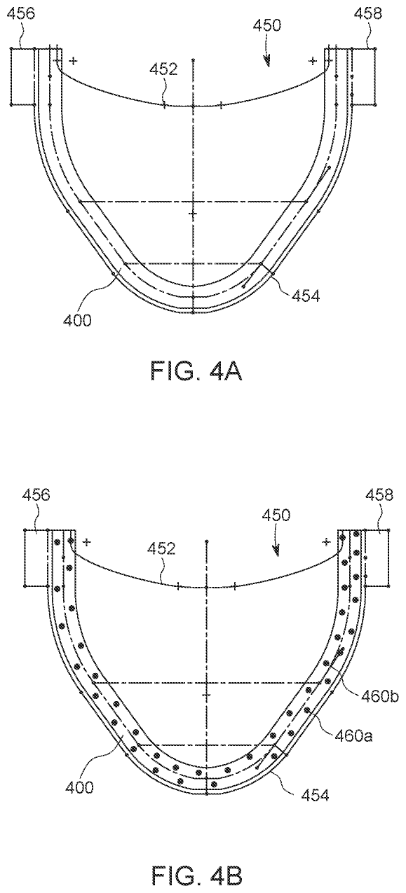

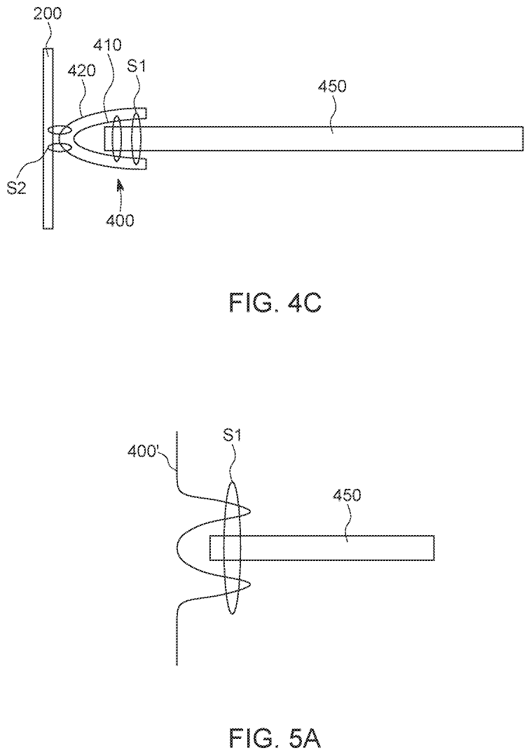

FIG. 4A illustrates a prosthetic leaflet 450 that may be used as part of a prosthetic heart valve, including in place of leaflets 250a-c described above. Leaflet 450 is shown in FIG. 4 in a flattened condition (e.g., on a table) prior to being coupled to other leaflets or to the frame of a prosthetic heart valve. The prosthetic leaflet 450 may be formed of any suitable material, including tissue (such as bovine or porcine pericardium) or synthetic materials, such as PET, PTFE, or UHWMPE. As shown in FIG. 4A, the prosthetic leaflet 450 may include a free edge 452 which is configured to move toward or away from other free edges of other prosthetic leaflets of a valve assembly to form the one-way valve. The prosthetic leaflet 450 may also include an attached edge 454 opposite the free edge 452. The attached edge 454 may be fixed to the frame of the prosthetic heart valve as described below, including in a manner that avoids direct coupling of the free edge 452 to the frame. The prosthetic leaflet 450 may also include tabs 456, 458 at side edges of the prosthetic leaflet 450, between the free edge 452 and the attached edge 454. The tabs 456, 458 may be used to attach one prosthetic leaflet 450 to an adjacent prosthetic leaflet 450. For example, if the prosthetic valve assembly includes three prosthetic leaflets 450, the tab 456 of each leaflet may be coupled to the tab 458 of an adjacent prosthetic leaflet 450. The commissures formed by the leaflet tabs 456, 458 may or may not be directly coupled to a commissure attachment feature of a frame of a prosthetic heart valve. For example, the leaflet commissures may be attached directly to the valve frame, directly to a woven material that is attached to the valve frame, or directly attached to a spacer fabric that is attached to the valve frame. The tabs 456, 458 may function, at least in part, as alignment features that assist in attaching the prosthetic leaflets 450 to the valve (e.g., the inner cuff of the valve) in a repeatable manner.

In the illustrated embodiment of FIG. 4A, the intermediate connection member 400 is coupled to the prosthetic leaflet 450. In this particular example, the intermediate connection member 400 follows the general contour of the attached edge 454 in a general “U”-shape or horseshoe shape. The intermediate connection member 400 does not extend over any substantial length (or any length at all) of the free edge 452, so as to not interfere with leaflet coaptation. Preferably, the intermediate connection member 400 is coupled to the prosthetic leaflet 450 in a “pre-assembly” step. In other words, the intermediate connection member 400 is preferably coupled to the prosthetic leaflet 450 before the prosthetic leaflet 450 is coupled to the valve frame, and before the prosthetic leaflet 450 is coupled to any other prosthetic leaflet 450.

In some embodiments, as shown in FIG. 4B, the prosthetic leaflet 450 may include apertures or holes that are formed at or adjacent to the attached edge 454 of the prosthetic leaflet 450. The apertures or holes may be formed by any suitable modality, including via the use of lasers. In the illustrated embodiment, the prosthetic leaflet 450 includes two rows of holes, including an outer row of holes 460a and an interior row of holes 460b. Each row of holes 460a, 460b may follow the general contour of the attached edge 454, which may be a “U”-shape or horseshoe shape. In the illustrated embodiment, the holes of the outer row 460a are staggered with the holes of the inner row 460b with respect to the direction of the contour of the attached edge 454. The holes 460a, 460b may be provided in other numbers of holes, numbers or rows of holes, and positions of holes besides those that are shown in FIG. 4B.

In typical prosthetic heart valves, the holes 460a, 460b may be utilized to directly couple the attached edge 454 of the prosthetic leaflet 450 to the frame of the prosthetic heart valve (and/or to an interior cuff or skirt on the luminal surface of the frame). However, in the illustrated embodiment, the holes 460a, 460b are instead used to suture, or otherwise couple, the intermediate attachment member 400 to the attached edge 454 of the prosthetic leaflet 450. By using these holes 460a, 460b, the design of the prosthetic leaflet 450 may be unchanged compared to typical prosthetic heart valves.

Referring to FIG. 4C, in order to attach the intermediate attachment member 400 to the attached edge 454 of the prosthetic leaflet 450, the intermediate attachment member 400 may be wrapped around or folded over the attached edge 454. After being folded over the attached edge 454, sutures S1 may be passed through the prosthetic leaflet 450 (e.g., via holes 460a, 460b) and through the intermediate attachment member 400 to fasten the intermediate attachment member 400 to the prosthetic leaflet 450. Preferably, this assembly step is performed prior to attaching the prosthetic leaflet 450 to another prosthetic leaflet, and before attaching the prosthetic leaflet 450 to the frame (e.g., frame 200) of the prosthetic heart valve. If the intermediate attachment member 400 takes the form of the spacer fabric 400 of FIGS. 3A-B, a particular arrangement of the spacer fabric 400 relative to the prosthetic leaflet 450 may be preferred. For example, as shown in FIG. 4C, the spacer fabric 400 may be folded over so that the finished surface 410 is facing and/or in contact with both faces of the prosthetic leaflet 450. After coupling the spacer fabric 400 to the prosthetic leaflet 450, the spacer fabric 400 may be attached to the frame 200 (and/or an inner cuff on the luminal surface thereof). For example, as shown in FIG. 4C, one or more sutures S2 may be used to pierce the spacer fabric 400 and wrap around the frame 200 (and/or the inner cuff). In the particular embodiment shown, the sutures S1 only pierce through the finished surface 410, but not through the mat surface 420, while sutures S2 only pierce through the mat surface 420, but not through the finished surface 410, of the spacer fabric 400. However, it should be understood that the spacer fabric 400 may be coupled to the frame 200 (and/or the inner cuff) via other suitable fasteners or modalities, including for example adhesives. It should be understood that, in the view of FIG. 4C, only a portion of the prosthetic leaflet 450 is illustrated, with the opposite main surfaces of the prosthetic leaflet 450 being on the top and bottom in the view of FIG. 4C, with the left side representing a portion of the attachment edge 454.

With the particular example illustrated in FIGS. 4A-C, and when the intermediate attachment member 400 takes the form of the spacer fabric 400 of FIGS. 3A-B, the inner matrix 430 and the finished surface 410 are able to move with the prosthetic leaflet 450 as the prosthetic leaflet 450 opens and closes during normal operation of the prosthetic heart valve. In other words, even if the frame 200 is formed as a rigid or stiff material, such as cobalt-chromium, deflection or other stress-reduction may be provided to the prosthetic leaflets 450 because the prosthetic leaflets 450 are not directly coupled to the frame 200. The particular configuration shown in FIG. 4C may enhance the deflection or stress reduction because the inner matrix 430 of the spacer fabric 400 may stretch as forces are applied during leaflet closing. Further, if the finished surface 410 and mat surface 420 are formed as a knit, the knit itself (particularly on the finished surface 410) may stretch as forces are applied during leaflet closing. This stress reduction on the prosthetic leaflet 450 may enable the prosthetic leaflets 450 to be coupled to a rigid frame 200 without any reduction in leaflet durability compared to prosthetic heart valves in which prosthetic leaflets are directly coupled to more flexible (e.g., nitinol) frames. It should be understood that the concepts described in connection with intermediate attachment member 400, and specifically the configuration of spacer fabric 400 shown in FIGS. 3A-B, may apply to other designs that differ from the specific spacer fabric 400 shown in FIGS. 3A-B. For example, the specific material(s) forming the intermediate attachment member 400, the thickness of any of the layers of the spacer fabric of FIGS. 3A-B, and specific weave or knit types may all be varied without departing from the scope of the invention. As described in greater detail below, the intermediate attachment member 400 (including the specific spacer fabric 400 shown in FIGS. 3A-B) may be impregnated with collagen prior to assembling the prosthetic heart valve to enable a pre-sealed fabric to be used in the design, reducing or eliminating the potential for leakages through the intermediate attachment member 400 (including the specific spacer fabric 400 shown in FIGS. 3A-B).

In another embodiment, a prosthetic heart valve similar or identical to that described in connection with FIGS. 1A-2B may include an inner cuff or skirt (e.g., similar to inner cuff 260) that is formed of the spacer fabric 400 of FIGS. 3A-B. In other words, the frame 200 may be formed from a rigid material such as cobalt-chromium, and the frame 200 may include an inner cuff 260 coupled to the luminal surface of the frame 200. The inner cuff 260, being formed from the spacer fabric 400 of FIGS. 3A-B, may have the finished surface 410 facing the frame 200, and the mat surface 420 facing the radial center of the frame 200. For example, sutures could pierce only the finished surface 410 to couple the inner cuff 260 to the frame 200. Instead of including an intermediate attachment member 400 on the prosthetic leaflet 450, the prosthetic leaflet 450 may be coupled to only the finished surface 410 of the spacer fabric 400 forming the inner cuff 260. For example, one or more sutures may pass through the holes (e.g., holes 460a, 460b) of the prosthetic leaflet, with those sutures only passing through the mat surface 420 of the spacer fabric 400 that forms the inner cuff 260. As a result, even though the prosthetic leaflet 450 is directly sutured to the inner cuff 260, similar deflection results as described in connection with the embodiment of FIGS. 4A-C. In other words, as the prosthetic leaflets 450 close and forces are applied to the prosthetic leaflets 450, the forces are transferred to the mat surface 420 of the spacer fabric 400, resulting in similar stretching and/or movement of the inner matrix 430 of the spacer fabric 400. This stretching or movement of the inner matrix 430, along with potential stretching of the knit of the finished surface 410 and/or the mat surface 420, better distributes the forces on the prosthetic leaflets 450, leading to a greater durability of the prosthetic leaflets 450, despite being used with a rigid frame. The variations described in connection with the spacer fabric 400 forming the intermediate attachment member 400 described above (e.g., including collagen impregnation and modified thicknesses, materials, and knits) may apply with equal force to the use of the spacer fabric 400 as the inner cuff 260. Further, as noted elsewhere, although these embodiments may be particularly suited to use with rigid (e.g., cobalt-chromium) frames, they may also be used with relatively flexible (e.g., nitinol) frames.

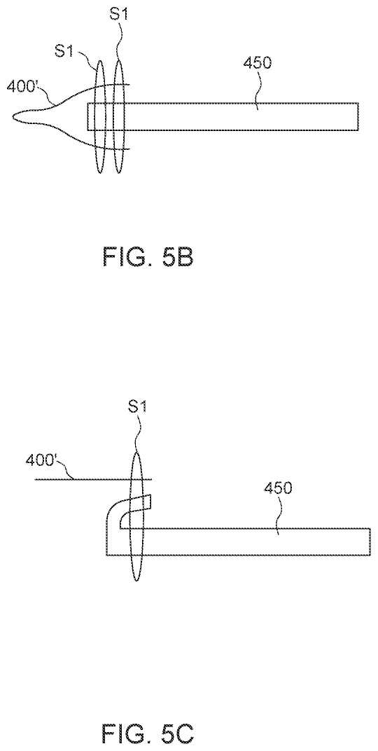

In another embodiment, the intermediate attachment member 400 shown in FIGS. 4A-B, instead of being the spacer fabric of FIGS. 3A-B, may be a woven fabric having the same general contour as shown in FIGS. 4A-B. However, instead of wrapping or folding the intermediate attachment member 400 as shown in FIG. 4C, other attachment configurations to the prosthetic leaflet 450 may be suitable. In each of the embodiments shown in and described in relation to FIGS. 5A-C, suture holes similar to holes 460a, 460b may be used if desired to couple the intermediate attachment member 400′ to the attachment edge 454 without needing to modify the prosthetic leaflet 450 configuration.

The woven fabric forming the intermediate attachment member 400′ may be coupled to the prosthetic leaflet 450 in different configurations, with examples shown in FIGS. 5A-C. For example, in FIG. 5A, the woven fabric forming the intermediate attachment member 400′ may be pinched to create two folds on either side of the attachment edge 454 of the prosthetic leaflet 450, and sutures S1 may be passed through the two folds and the leaflet (e.g., holes 460a, 460b) to attach the woven fabric along the contour of the attachment edge 454. FIG. 5B illustrates an alternate configuration in which the woven fabric 400′ may be folded around the attachment edge 454 of the prosthetic leaflet 450, in a generally similar fashion as described in connection with FIG. 4C. However, in FIG. 5B, the fold of the woven fabric 400′ may create two fabric layers that are in close contact with each other, which may be slightly different than the spacer fabric 400 shown in FIG. 4C. In the embodiment shown in FIG. 5C, the attachment edge 454 of the prosthetic leaflet 450 may be folded over itself, and the woven fabric 400′ sutured through both layers of the prosthetic leaflet 450 at the folded attachment edge 454. With any of the embodiments shown in, and described in connection with, FIGS. 5A-C, the woven fabric 400′ may first be attached along the contour of the free edge 454 of the prosthetic leaflet 450 prior to coupling the prosthetic leaflets 450 to each other or to the frame (and/or inner cuff) of the prosthetic heart valve. After assembling the woven fabric 400′ to the prosthetic leaflet 450, the woven fabric 400′ may then be attached, e.g., via suturing, to an inner cuff of the prosthetic heart valve. For example, the inner cuff may be the spacer fabric 400, and in a similar fashion as described in the paragraphs above, the woven fabric 400′ may be attached only to the mat surface 420 of the spacer fabric 400 that forms the inner cuff, with the finished surface 410 being attached to the frame. However, in other embodiments, the woven fabric 400′ may be coupled to an inner cuff formed of a more traditional woven fabric. In some embodiments, parameters of the woven fabric 400′, such as thread diameter, weave style, ends per inch, and picks per inch, may be adjusted to create additional deflection. These configurations described above would result in a reduction in the stress on the prosthetic leaflets 450, particularly during leaflet closing, in a similar or the same fashion as described in the embodiments above. It should be understood that, in the views of FIGS. 5A-5C, only a portion of the prosthetic leaflet 450 is illustrated, with the opposite main surfaces of the prosthetic leaflet 450 being on the top and bottom in the views of FIGS. 5A-C, with the left side representing a portion of the attachment edge 454.

Generally, it is important that after the implantation of a prosthetic heart valve, blood only flows through the prosthetic heart valve via the prosthetic leaflets when they are open, and no flow across the prosthetic heart valve occurs when the prosthetic leaflets are closed. In practice, flow around the outside of the prosthetic heart valve, which may be referred to as PV leakage, can occur as a result of suboptimal fit between the prosthetic heart valve and the native valve annulus. For example, as described above and shown in FIG. 1K, a prosthetic heart valve incorporating stent 200 may be provided with an outer skirt or cuff 270 that is configured to contact the native valve annulus to help enhance sealing against PV leak. Although outer cuff 270 may be formed from various materials, in some embodiments the outer cuff 270 is formed of a synthetic material, such as woven UHMWPE. As described in connection with FIG. 1K, the fabric outer cuff 270 may be coupled (e.g., fastened via sutures) to the abluminal surface of the stent 200 to create pockets that can actively open (during ventricular diastole) and close (during ventricular systole) to reduce PVL. After implantation of the prosthetic heart valve, tissue ingrowth will typically occur over time into the outer cuff 270 to help more permanently seal any space or gaps between the outer cuff 270 and the inside of the native valve annulus. However, this natural sealing may not always occur effectively. In some scenarios, a patient may have one or more large calcium nodules in or on the native valve annulus or native leaflets. These calcium nodules may result in areas of low or no contact between the prosthetic heart valve and the patient's tissue, which may reduce the ability of tissue to effectively grow into the outer cuff 270 to completely seal gaps between the prosthetic heart valve and the native tissue. One mechanism to help alleviate this potential non-sealing is to impregnate the outer cuff 270 with collagen. And while it may be helpful to impregnate the outer cuff 270 with collagen, it may also be helpful to additionally or alternately impregnate the inner cuff 260 with collagen. By impregnating the inner cuff 260 and/or outer cuff 270 with collagen, the cuff(s) may be pre-sealed (e.g., fabric porosity reduced upon implantation) to help prevent PV leak, particularly in the event that the cuff(s) does not completely endothelialize (or plug), during the lifetime of the prosthetic valve implantation.

Similar to impregnating inner and/or outer cuffs formed of woven UHMWPE or similar materials as described above, the spacer fabric 400, as shown in FIGS. 3A-B, may be impregnated with collagen for pre-sealing in a similar fashion, whether the spacer fabric 400 is used as an outer cuff, and inner cuff, or an intermediate leaflet attachment. The configuration of the spacer fabric may provide bulk and stretch to the fabric design, while being able to compress into a thin profile for loading (e.g., crimping onto a balloon for delivery). As described above, the spacer fabric 400 includes an inner matrix 430 of fiber connecting the mat side 420 and the finished side 410. The inner matrix 430 may provide an opportunity for blood to pass through the inner matrix 430, which may result in leakage. However, by impregnating the spacer fabric 400 with collagen, the spacer fabric 400, including the inner matrix 430, is pre-sealed and allows for immediate resistance against leakage, compared to tissue ingrowth naturally creating resistance against leakage over a longer time period (or tissue ingrowth failing to occur as a result of calcium nodules or any other reason).

It should be understood that although collagen impregnation of a fabric used in a prosthetic heart valve is described above in the context of specific inner and/or outer cuffs formed of woven UHMWPE and in the context of spacer fabrics for use as inner cuffs, outer cuffs, and/or intermediate leaflet attachment members, the invention is not so limited. In other words, other fabric materials for use in prosthetic heart valves other than those specifically described above may be impregnated with collagen prior to implantation to assist in pre-sealing the fabric. Such collagen impregnation typically will not inhibit the ability of the material to compress, or the ability of the material to continue to press outwards into the native annulus around calcium nodules or into void space when used as an outer or inner cuff.

When deploying a balloon-expandable prosthetic heart valve into a native valve annulus, it may be important for the prosthetic heart valve to be in a particular location prior to beginning to inflate the balloon, and it may also be important that the prosthetic heart valve expands in a controlled and/or predictable manner Various features described below may assist with achieving one or more of these goals. Although the below description is provided in the context of balloon-expandable prosthetic aortic valves, it should be understood that these features may be employed with other balloon-expandable heart valves.

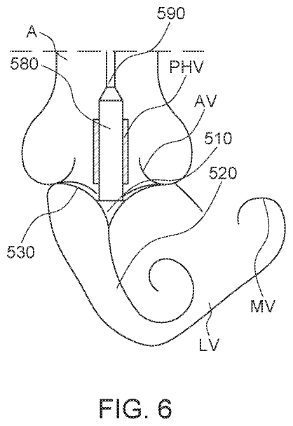

FIG. 6 illustrates a distal end portion of a delivery device having been delivered via a transfemoral route and through a patient's aorta A into the aortic valve AV. FIG. 6 omits from the illustration the left atrium, but shows the mitral valve MV separating the left atrium from the left ventricle LV. The delivery device may include an atraumatic distal tip 510 and may be delivered over a guidewire 520. The delivery device may include a balloon catheter 590 which may be generally similar to that described in connection with FIGS. 2A-B, or similar to any other suitable known balloon catheter. In FIG. 6, a prosthetic heart valve PHV has been crimped onto a balloon 580, which is shown in FIG. 6 as still being deflated, prior to delivery of the prosthetic heart valve PHV to the aortic valve AV. The prosthetic heart valve PHV may be similar to any prosthetic heart valve described herein, or any other suitable balloon-expandable prosthetic heart valve.

A unique feature shown in connection with FIG. 6 is a position locator 530 at or near a distal end of the delivery device, for example at a position distal to the leading edge of the prosthetic heart valve PHV. The position locator 530 may be formed as a braided structure (e.g., a braided mesh, including a braided nitinol mesh) or as one or more individual wires (e.g., nitinol wires). In the illustrated example, the position locator 530 includes two wires positioned at substantially opposite diametric sides of the delivery device. However, more than two wires could be provided if the position locator 530 is formed as wires. If the position locator 530 is formed as a braid, the braid may substantially circumscribe the balloon catheter 590. Preferably, the position locator 530 has a first end coupled to the delivery device, for example to the shaft of the balloon catheter 590 distal to the leading edge of the prosthetic heart valve PHV or to a proximal end of the atraumatic distal tip 510. The position locator 530 preferably extends in a proximal direction from the first end to a second free end. During delivery of the prosthetic heart valve PHV, prior to reaching the aortic valve AV, the position locator 530 may be kept in a stowed or closed condition, for example in which the position locator 530 overlies portions of the prosthetic heart valve PHV, including the leading edge of the prosthetic heart valve PHV. With this configuration, the position locator 530 may help protect the leading edge of the prosthetic heart valve PHV from contacting tissue, for example as the device tracks around the aortic arch, which contact could otherwise lead to an undesirable repositioning of the prosthetic heart valve PHV on the balloon 580. In some embodiments, the position locator 530 may be formed from a shape memory material and be set to the stowed or closed condition in the absence of applied forces. In other embodiments, the position locator 530 may be formed from a shape memory material and be set to an open or activated condition (described in more detail below), with one or more suture wires coupled to the position locator 530 to maintain the position locator 530 in the stowed condition during delivery. In some embodiments, the position locator 530 may be stowed inside the distal tip 510 and/or the balloon catheter 590 at a location distal to the balloon 580 and deployed using an internal actuation mechanism such that wires or a braid are advanced outside of the balloon catheter 590 when desired by actuation of a proximal control handle and attain the desired shape and position via shape memory properties.

In use, the delivery device of FIG. 6 may be delivered toward the aortic valve AV, for example over guidewire 520, while the position locator 530 is either actively or passively maintained in the stowed condition and providing protection to the leading edge of the prosthetic heart valve PHV crimped on the balloon 580. In this stowed condition, the position locator 530 has a relatively small radial extent or diameter such that the position locator 530 may relatively easily pass through the aortic valve AV when the native aortic valve leaflets are open (as shown in FIG. 6). Once the delivery device has been advanced at least partially through the native aortic valve AV, the user may transition the position locator 530 to a deployed, activated, or expanded condition, as shown in FIG. 6. The position locator 530 may be transitioned to the deployed, activated, or expanded condition by any suitable mechanism. For example, if the position locator 530 is formed of shape memory material and set to the deployed condition, with sutures or other mechanisms maintaining the position locator 530 in the stowed condition during delivery, tension on the sutures may be released to allow the position locator 530 to expand or activate. If the position locator 530 is formed of shape memory material and set to the stowed condition, a different mechanism may be used to transition the position locator 530 to the deployed or activated condition. For example, one or more sutures may run through an interior shaft of the delivery device toward the atraumatic distal tip 510, and loop proximally to connect to the free end(s) of the position locator 530. In this configuration, pulling the sutures proximally will force the position locator 530 to activate.

Regardless of the particular mechanism of activation, once the position locator 530 is activated, the position locator 530 has a radial extent or diameter that is too large to easily pass through the native aortic valve AV. While the position locator 530 is in the activated or deployed condition, the delivery device may be pulled proximally until the free end(s) of the position locator 530 contact the ventricular side of the annulus of the native aortic valve AV, confirming the axial location of the delivery device (and thus the axial location of the prosthetic heart valve PHV) relative to the annulus of the native aortic valve AV. Once positioning is confirmed, the balloon 580 may be inflated to force the prosthetic heart valve PHV to expand into the native aortic valve AV. Because the position of the prosthetic heart valve PHV relative to the position locator 530 is known, and because the position of the position locator 530 relative to the native aortic valve AV is known, the prosthetic heart valve PHV will be able to be consistently positioned in the optimal position relative to the native aortic valve AV.

In some embodiments, the position locator 530 may be moved after the desired position is confirmed to ensure that the position locator 530 does not interfere with the expansion of the balloon 580 and the prosthetic heart valve PHV. For example, if sutures were pulled proximally to activate the position locator 530, after the desired position is confirmed, the sutures may be pulled further to move the position locator 530 farther out of the way to help ensure it does not interfere with expansion of the prosthetic heart valve PHV. In other embodiments, the position locator 530 may be coupled to a separately moveable shaft that may be translated distally to move the position locator 530 further into the left ventricle LV prior to expansion of the prosthetic heart valve PHV.

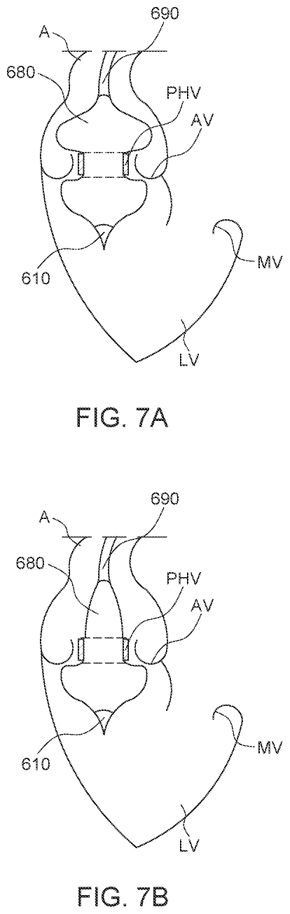

FIG. 7A illustrates a distal end portion of a delivery device having been delivered via a transfemoral route and through a patient's aorta A into the aortic valve AV, in a generally similar position as described and shown in connection with FIG. 6. The delivery device may include an atraumatic distal tip 610 and may be delivered over a guidewire (not shown in FIG. 7A). The delivery device may include a balloon catheter 690 which may be generally similar to that described in connection with FIGS. 2A-B and FIG. 6, or similar to any other suitable known balloon catheter. In FIG. 7A, a prosthetic heart valve PHV has been crimped onto a balloon 680.

In FIG. 7A, the balloon 680 has already begun to undergo a first stage of expansion. The result of the first stage of expansion is that a portion of the balloon 680 distal to the prosthetic heart valve PHV and a portion of the balloon 680 proximal to the prosthetic heart valve PHV have at least partially expanded to a larger diameter, while the portion of the balloon 680 which the prosthetic heart valve PHV has not begun to expand (or to significantly expand). With this configuration, during the first stage of expansion, if the prosthetic heart valve PHV is properly axially aligned with the native aortic valve AV, the proximal portion of the balloon 680 and the distal portion of the balloon 680 will each expand on opposite sides of the native aortic valve AV. If the prosthetic heart valve PHV is not properly axially aligned with the native aortic valve AV, the user can advance or retract the delivery device and start (or continue) to perform the first stage of balloon expansion until the configuration shown in FIG. 7A is achieved. With two portions of the balloon 680 expanded on opposite sides of the native aortic valve AV, the proximal and distal end portions of the balloon 680 may act as a positioning aid, effectively ensuring that the prosthetic heart valve PHV is, and remains, axially aligned with the native aortic valve AV once the proximal and distal portions of the balloon 680 are expanded during the first stage of expansion.

While the balloon 680 is in the first stage of expansion 680 shown in FIG. 7A, the user may begin a second stage of expansion in which the center portion of the balloon 680 expands to force the prosthetic heart valve PHV to expand into the native aortic valve AV. Because the proximal and distal portions of the balloon 680 maintain the desired position of the balloon 680 relative to the native aortic valve AV, the prosthetic heart valve PHV will be axially aligned with the native aortic valve AV during the entirety of the second stage of expansion, helping to ensure the desired positioning.

Any one or more of various mechanisms may be selected to help allow for the two different stages of expansion to occur. For example, the proximal and distal portions of balloon 680 may be formed from a first material and the central portion of the balloon 680 may be formed from a second different material balloon materials may be selected so that, upon an initial push of fluid to the balloon 680, the first material preferentially begins to expand until it reaches the desired size, while the second material does not begin to expand until after the proximal and distal portions of the balloon 680 have already expanded. Alternately, or additionally, the proximal and distal portions of balloon 680 may be formed with a first wall thickness, while the central portion of the balloon 680 is formed with a second wall thickness, which may be greater than the first wall thickness.

Although FIG. 7A illustrates an embodiment in which the balloon 680 includes both proximal and distal portions that expand in a first stage of expansion prior to a center portion of the balloon 680 expanding the prosthetic heart valve PHV, an alternate embodiment may work similarly, as shown in FIG. 7B. Rather than have both proximal and distal portions 680 of the balloon expand initially in a first stage of expansion, only a distal portion of the balloon 680 may expand during a first stage of expansion, as shown in FIG. 7B. In this embodiment the proximal and/or center portion of the balloon 680, over which the prosthetic heart valve PHV is crimped, does not expand during the first stage of expansion. Rather, the distal expanded portion of the balloon 680 may be used to confirm positioning of the prosthetic heart valve PHV, with the expanded distal portion of the balloon 680 in contact with the ventricular side of the native aortic valve AV. Otherwise, the methods and structures described in connection with FIG. 7A may apply with substantially equal force to FIG. 7B. In some embodiments, the balloon 680 may be provided as two separate balloons, so that the first balloon positioned distally is expanded first via a first inflation lumen, and the center and/or proximal balloon, which is a separate second balloon, is expanded second via a separate second inflation lumen. Alternatively, the same approach could be taken to initially expand only the proximal portion of the balloon if desired.

And while FIGS. 7A-B generally describe that the balloon 680 forms one or more shoulder-type structures after some amount of inflation, it should be understood that the balloon 680 may instead (or in addition) be heat-set or otherwise formed so that pillows or shoulders are formed on one or both sides of the prosthetic heart valve PHV prior to any significant expansion of the balloon 680, for example as described in greater detail in U.S. Provisional Patent Application No. 63/382,812, filed Nov. 8, 2022 and titled “Prosthetic Heart Valve Delivery and Trackability,” the disclosure of which is hereby incorporated by reference herein. In such examples, the inflow edge of the prosthetic heart valve may be covered and/or protected by a pillowed shape or shoulder shape of the balloon during delivery. Similar to what is shown in FIG. 7B, the pillow or shoulder may be used as a datum (e.g. by contacting the inflow side of the aortic valve annulus and/or the inflow side of the native aortic valve leaflets) to help maintain the position of the inflow edge of the prosthetic heart valve during expansion. As expansion of the balloon 680 continues, the resulting deployed position of the prosthetic heart valve is such that the inflow end of the prosthetic heart valve does not shift axially (or does not shift axially to a significant extent) between just prior to expansion of the balloon 680 up to and including full expansion of the balloon 680 and full deployment of the prosthetic heart valve.

While it may be useful to help ensure a desired axial position of a prosthetic heart valve relative to a native heart valve prior to deploying the prosthetic heart valve via balloon expansion, it may also be useful to have features that assist in creating general uniform radial expansion of the prosthetic heart valve via the balloon. In other words, even if a prosthetic heart valve has desired axial positioning prior to deployment, if the prosthetic heart valve does not generally uniformly expand via the balloon, the placement of the prosthetic heart valve within the native heart valve may be suboptimal.

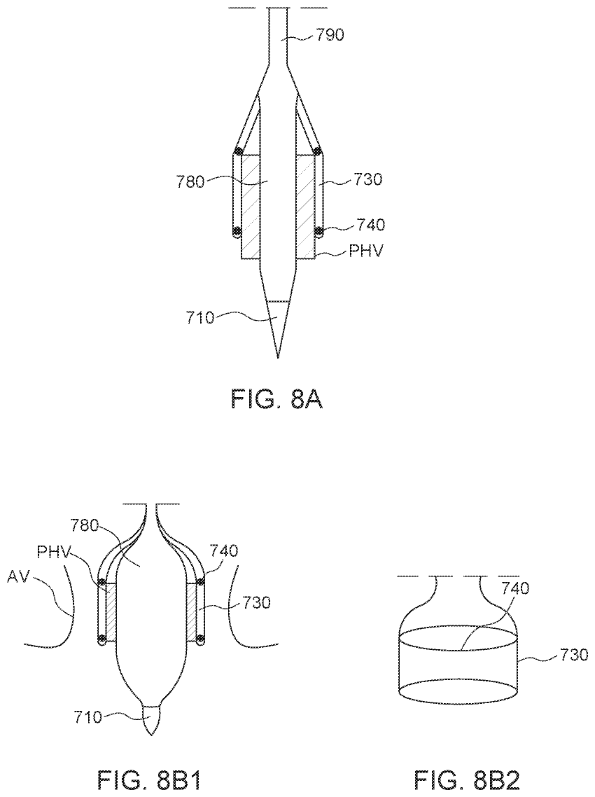

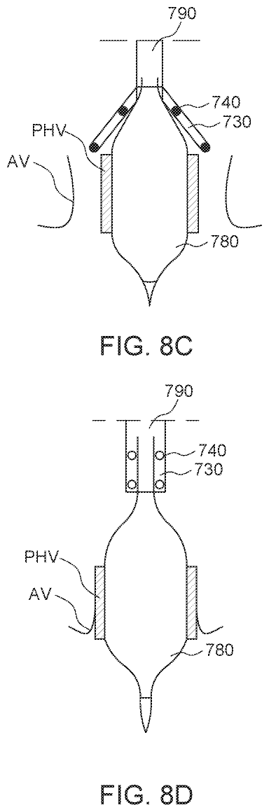

FIGS. 8A-D illustrate an example of a feature that may assist with creating generally uniform radial expansion of a prosthetic heart valve via balloon expansion. FIG. 8A illustrates the distal end of a delivery device, showing a balloon catheter 790 and a prosthetic heart valve PHV crimped over a balloon 780 of the balloon catheter 790, with the balloon 780 in a deflated condition. As shown in FIG. 8A, the delivery device may include one or more expandable members 730 having one or more expansion limiters 740. In the particular illustrated example, the expandable members 730 are two individual arms that extend distally from the balloon catheter 790 in a direction toward atraumatic distal tip 710 and radially outwardly of the prosthetic heart valve PHV so that the distal free ends of the expandable members 730 are positioned at or adjacent to the leading edge of the prosthetic heart valve PHV. Although two expandable members 730 are shown at generally opposite diametrical positions, more than two expandable members 730 may be provided, including three, four, etc., preferably at substantially equal intervals in a circumferential direction. The expandable members 730 may extend proximally and may be attached to a separately translatable shaft so that the expandable members 730 may be retracted by pulling the separate shaft proximally. That separate shaft may be positioned radially inwardly of an outer sheath of the delivery device, including, for example, balloon catheter 790. Each expandable member 730 may include one or more through holes for receiving expansion limiters 740, which may be wires, sutures, or similar structures. Although the wires or sutures 740 are shown as extending through the through holes of the expandable members 730, in other embodiments the expansion limiters 740 may be coupled to the expandable members 730 in different ways (e.g., adhesives, knotting, or fasteners).

The prosthetic heart valve PHV is delivered toward the native aortic valve AV while crimped over the balloon 780 and with the expandable members 730 overlying the crimped prosthetic heart valve PHV on the deflated balloon 780. Once the prosthetic heart valve PHV is at the desired location relative to the native aortic valve AV, as shown in FIG. 8B1, a user may begin to expand the balloon 780, for example by pushing fluid into the balloon 780. Notably, the balloon 780 is not fully expanded at this stage, but rather only partially expanded. As the balloon 780 expands, the expandable members 730 are also forced radially outwardly, until the expansion limiters 740 achieve a maximum diameter. FIG. 8B2 shows the view of FIG. 8B1 with all components other than the expandable members 730 and the expansion limiters 740 omitted. Referring to FIGS. 8B1 and 8B2, the balloon 780 may be expanded until the expansion limiters 740 reach a generally circular configuration at their maximum diameter, at which point the expansion limiters 740 will resist further expansion of the balloon 780. At the partial expansion shown in FIG. 8B1, neither the prosthetic heart valve PHV nor the expandable members 730 are in contact with tissue of the native aortic valve AV. In the illustrated condition, the balloon 780 and prosthetic heart valve PHV are generally forced to take a cylindrical shape due to the restrictions of the expansion limiters 740. In other words, the expansion limiters 740 help ensure that the prosthetic heart valve PHV, at this mid-stage of expansion, takes no shape other than a generally cylindrical shape.

It should be understood that, as the prosthetic heart valve PHV is expanded, depending on the particular design of the prosthetic heart valve PHV, it may axially foreshorten. In other words, the relative axial positioning between the prosthetic heart valve PHV and the native aortic valve AV may shift slightly during expansion of the prosthetic heart valve PHV into the aortic valve AV. Thus, once the prosthetic heart valve PHV has been partially expanded, as shown in FIG. 8B1, it may be desirable to confirm (e.g., via fluoroscopy or other imaging) that the axial positioning relative to the native aortic valve AV is still desirable. If the positioning is not desirable, the delivery catheter may be advanced or retracted to obtain the desired positioning. Whether or not the relative axial positioning is re-confirmed or adjusted, prior to fully expanding the prosthetic heart valve PHV into the native aortic valve AV, the expandable members 730 and expansion limiter 740 may be withdrawn so that they no longer limit expansion. For example, as shown in FIG. 8C, while the balloon 780 remains in a partially expanded state, a separate shaft to which the expandable members 730 are coupled may be withdrawn proximally to draw the expandable members 730 at least partially inside the balloon catheter 790 (or a separate overlying catheter). The expandable members 730 may be withdrawn partially or completely into the balloon catheter 790 (or the separate overlying catheter), as long as the free ends of the expandable members 730 (and all of the expansion limiters 740) are clear of the trailing edge of the prosthetic heart valve PHV. In some embodiments, the expandable members 730 and expansion limiter 740 may be collapsed into the balloon catheter 790 (or separate overlying catheter) while intact, but in other embodiments, they may be ripped, broken, and/or partially detached to enable the withdrawal.

As shown in FIG. 8D, with the expandable members 730 and the expansion limiters 740 stowed within the balloon catheter 790 (or a separate overlying catheter), the balloon 780 may be further expanded so that the prosthetic heart valve PHV is deployed into the native aortic valve AV. Because the prosthetic heart valve PHV had already been partially expanded in a radially uniform manner in the previous step, the remaining expansion of the prosthetic heart valve PHV is more likely to continue to be radial uniform expansion, and any axial foreshortening of the prosthetic heart valve PHV may have already been compensated for prior to the step of complete expansion. With the prosthetic heart valve PHV deployed, the balloon 780 may be deflated and the delivery device may be withdrawn from the patient to complete the procedure.

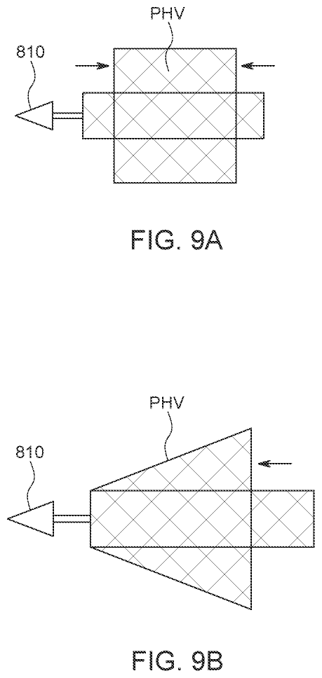

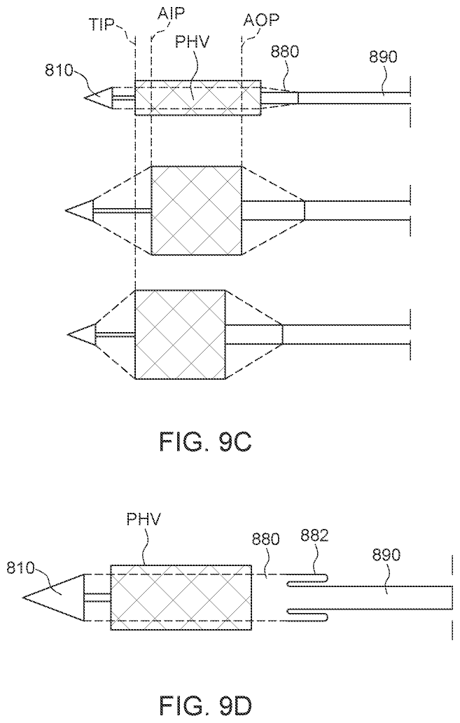

FIG. 9A illustrates a balloon-expandable prosthetic heart valve PHV on a balloon (not separately labeled) of a delivery device, including an atraumatic distal tip 810. In FIG. 9A, the prosthetic heart valve PHV is illustrated in two conditions, including the crimped condition, and also the expanded condition. FIG. 9A illustrates the point above that when the prosthetic heart valve PHV expands, it may axially foreshorten, as indicated by the two arrows indicating the length difference. As noted above, this may be undesirable because the relative positioning between the prosthetic heart valve PHV and the native aortic valve AV changes as the prosthetic heart valve PHV expands. One way to reduce the potential negative effects of axial foreshortening is to control the directionality of expansion. In other words, in the embodiment of FIG. 9A, all axial portions of the prosthetic heart valve PHV expand at the same time. However, if the proximal end (which is the outflow end for a transfemoral delivery) of the prosthetic heart valve PHV is expanded first, most of all of the axial foreshortening will tend to occur at the outflow end of the prosthetic heart valve PHV, as indicated by the arrow in FIG. 9B, while the distal end (which is the inflow end for a transfemoral delivery) of the prosthetic heart valve PHV will tend to foreshorten less. In other words, in a transfemoral delivery of a balloon-expandable prosthetic heart valve PHV, the distal or inflow end of the prosthetic heart valve PHV may be generally axially aligned with the desired position of the native aortic valve AV. If the balloon was expanded uniformly, the inflow end of the prosthetic heart valve PHV might shift slightly proximally, potentially leading to suboptimal positioning. However, if the proximal end of the balloon is expanded first, the outflow end of the prosthetic heart valve PHV will expand first and axially foreshorten, as shown in FIG. 9B. Then, balloon expansion can be continued, so that the inflow end of the prosthetic heart valve PHV expands with little or no change in relative axial positioning relative to the desired axial position of the native aortic valve AV. To achieve this ordered inflation, two separate balloons may be used so that a proximal balloon is inflated first, and then a distal balloon is inflated second. In another embodiment, a single balloon with a thinner proximal wall and thicker proximal wall may be used to obtain the ordered expansion. In some embodiments, the surface of the balloon may be provided with texturing or other friction-increasing features at the distal end of the balloon. With the proximal end of the balloon having less friction than the distal end of the balloon, as the balloon expands, the proximal end of the prosthetic heart valve PHV will tend to foreshorten while the distal end of the prosthetic heart valve PHV will tend to resist foreshortening because of the added friction.

FIG. 9C illustrates the concept described in connection with FIG. 9A. For example, FIG. 9C illustrates a prosthetic heart valve PHV collapsed over the balloon 880 of the balloon catheter 890 just prior to expansion. In the top view of FIG. 9C, the inflow end of the prosthetic heart valve PHV is aligned with a target inflow position TIP of the native aortic valve. However, if the axial foreshortening is not compensated for, as shown in the middle view of FIG. 9C, the actual inflow position AIP of the prosthetic heart valve PHV, after expansion, has shifted from the target inflow position TIP. Similarly, the actual outflow position AOP has shifted from the position of the outflow end of the prosthetic heart valve PHV when crimped. It should be understood that it is typically more important to maintain the inflow end of the prosthetic heart valve PHV at the intended position relative to the native aortic valve annulus, than it is to ensure that the outflow end of the prosthetic heart valve PHV does not shift its axial position. It may be desirable to allow for compensation of the axial foreshortening, as shown in the bottom view of FIG. 9C, which shows the inflow end of the prosthetic heart valve PHV being aligned with the initial target inflow position TIP despite axial foreshortening during expansion. Although the outflow end of the prosthetic heart valve PHV in the bottom view of FIG. 9C has shifted from the original positioning, as noted above, that is not particularly problematic.

One way to automatically compensate for the axial foreshortening is shown in FIG. 9D. In particular, the balloon 880 of balloon catheter 890 in FIG. 9D includes a folded proximal portion or neck 882. In other words, in the collapsed or deflated condition shown in FIG. 9D, the proximal folded portion 882 of the balloon 880 extends proximally from the point at which the balloon 880 couples to the balloon catheter 890. As a result, as the balloon 880 is inflated and the folded portions 882 of the balloon 880 unfold, the position of the balloon 880 will shift slightly distally as the prosthetic heart valve PHV expands. The size of the fold may dictate, at least in part, the distance which the prosthetic heart valve PHV is advanced axially during expansion of the balloon 880. Preferably, the fold 882 is sized and positioned so that, as the prosthetic heart valve PHV expands via expansion of the balloon 880, the distal axial shift of the prosthetic heart valve PHV is about the same as the difference between the target inflow position TIP in the top view of FIG. 9C and the actual inflow position AIP in the middle view of FIG. 9C. In other words, the inclusion of the fold 882 in the balloon 880 will automatically compensate for the axial foreshortening, allowing the inflow end of the prosthetic heart valve PHV to be deployed into the target inflow position TIP without any special additional adjustment, achieving the positioning shown in the bottom view of FIG. 9C. In other examples, a mechanism may be provided at the handle of the delivery device to actuate an inner sheath and/or nosecone to move the inner sheath and/or nosecone distally as the balloon expands. For example, U.S. Provisional Patent Application No. 63/343,479, filed May 18, 2022, and titled “Automated Balloon Inflation Device for Transcatheter Heart Valve Implantation,” describes a delivery device that automates the inflation of the balloon. The contents of that application are hereby incorporated by reference herein. As the balloon expands, and the delivery device tracks the amount of balloon inflation in real-time, upon reaching a particular inflation threshold, the mechanism in the delivery device may advance the inner shaft and/or nosecone of the delivery device distally to move the balloon and thus the prosthetic heart valve PHV distally as it expands to account for the change in distance caused by the foreshortening.

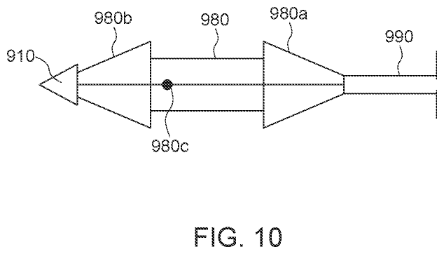

In some embodiments, one or more radiopaque markers may be employed to assist with helping to ensure the desired axial positioning of the prosthetic heart valve PHV relative to the native aortic valve, particularly to account for axial foreshortening. For example, the distal end of a delivery device is shown in FIG. 10, which includes a balloon catheter 990 extending to an atraumatic distal tip 910. In some embodiments, the balloon 980 may include one or more radiopaque tapers 980a, 980b, to help visualize the position of the balloon 980 relative to the patient anatomy. For example, FIG. 10 illustrates a proximal taper 980a, and a distal taper 980b, which may be easily visualized (e.g., during fluoroscopy) to correctly position the balloon 980 and the prosthetic heart valve PHV relative to the anatomy. Additionally or alternatively, a radiopaque marker 980c may be provided on an interior shaft of the delivery device. Radiopaque marker 980c may indicate the expected position that the inflow end of the prosthetic heart valve PHV will be positioned after balloon expansion and axial foreshortening of the prosthetic heart valve PHV. For example, referring again to FIG. 9C, the radiopaque marker 980c may be positioned at the actual inflow position AIP shown in the middle view of FIG. 9C. With this configuration, the radiopaque marker 980c, instead of the inflow end of the crimped prosthetic heart valve PHV, may be aligned with the desired positioning in the native aortic valve AV. Thus, as the balloon 990 is inflated and the prosthetic heart valve PHV expands and axially foreshortens, the inflow end of the prosthetic heart valve PHV will, after expansion, be generally aligned with radiopaque marker 980c and thus the desired axial position with respect to the native aortic valve AV.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims. Further, it should be understood that different embodiments described herein may be combined with other embodiments described herein to achieve the benefits of both embodiments.

1. A prosthetic heart valve, comprising:
a collapsible and expandable frame; and
a plurality of prosthetic leaflets, each of the prosthetic leaflets having a free edge, an opposite attached edge having a contour, a first flat surface facing toward the frame, and a second flat surface facing away from the frame; and
a spacer fabric is attached to each of the prosthetic leaflets, the spacer fabric having a first surface, a second surface, and an inner matrix between the first surface and the second surface;
wherein each spacer fabric follows the contour of the attached edge of a corresponding one of the prosthetic leaflets, and each spacer fabric is wrapped around the contour of the attached edge of the corresponding one of the prosthetic leaflets such that the first surface of each spacer fabric contacts the first flat surface and the second flat surface of the corresponding prosthetic leaflet, each prosthetic leaflet being coupled to the frame via the corresponding spacer fabric.

2. The prosthetic heart valve of claim 1, wherein the first surface of each spacer fabric is sutured to the corresponding prosthetic leaflet.

3. The prosthetic heart valve of claim 2, wherein the second surface of each spacer fabric is not directly coupled to the corresponding prosthetic leaflet.

4. The prosthetic heart valve of claim 2, wherein the second surface of each spacer fabric is sutured to the frame.

5. The prosthetic heart valve of claim 4, wherein the first surface of each spacer fabric is not directly coupled to the frame.

6. The prosthetic heart valve of claim 1, wherein the first surface of each spacer fabric is a finished surface, and the second surface of each spacer fabric is a mat surface.

7. The prosthetic heart valve of claim 1, wherein the spacer fabric is impregnated with collagen.

8. A method of implanting a prosthetic heart valve, the method comprising:
advancing a delivery device to a native aortic valve of a patient while the prosthetic heart valve is crimped over a balloon of the delivery device;
while the prosthetic heart valve remains crimped over the balloon, inflating a distal portion of the balloon positioned on a ventricular side of the native aortic valve;
wherein inflating the distal portion of the balloon does not expand the prosthetic heart valve into contact with the native aortic valve;
after inflating the distal portion of the balloon, confirming that the inflated distal portion of the balloon is in contact with the ventricular side of the native aortic valve; and
after confirming, inflating a second portion of the balloon to expand the prosthetic heart valve into contact with the native aortic valve.

9. The method of claim 8, further comprising inflating a proximal portion of the balloon positioned on an aortic side of the native aortic valve before inflating the second portion of the balloon, so that the native aortic valve is sandwiched between the inflated proximal portion of the balloon and the inflated distal portion of the balloon.

10. The method of claim 9, wherein inflating the proximal portion of the balloon on the aortic side of the native aortic valve and inflating the distal portion of the balloon on the ventricular side of the native aortic valve are performed simultaneously.

11. The method of claim 9, wherein the second portion of the balloon is a central portion of the balloon.

12. The method of claim 11, wherein prior to expanding the prosthetic heart valve into contact with the native aortic valve, the prosthetic heart valve is crimped over the central portion of the balloon.

13. The method of claim 11, wherein the proximal portion of the balloon and the distal portion of the balloon are each formed from a first material, and the central portion of the balloon is formed from a second material that is different from the first material so that, upon an initial push of fluid to the balloon, the first material preferentially begins to expand before the second material begins to expand.

14. The method of claim 11, wherein the proximal portion of the balloon and the distal portion of the balloon are each formed with a wall thickness that is smaller than a wall thickness of the central portion of the balloon.

15. The method of claim 8, wherein inflating the distal portion of the balloon centers the prosthetic heart valve within the native aortic valve.

16. The method of claim 8, wherein prior to confirming that the inflated distal portion of the balloon is in contact with the ventricular side of the native aortic valve, it is determined that the prosthetic heart valve is not axially aligned with the native aortic valve.

17. The method of claim 16, further comprising, after determining that the prosthetic heart valve is not axially aligned with the native aortic valve and prior to confirming that the inflated distal portion of the balloon is in contact with the ventricular side of the native aortic valve, advancing or retracting the delivery device until the prosthetic heart valve is axially aligned with the native aortic valve

18. The method of claim 16, wherein during inflating the second portion of the balloon to expand the prosthetic heart valve into contact with the native aortic valve, the prosthetic heart valve maintains consistent axial alignment with respect to the native aortic valve.

19. The method of claim 8, wherein upon inflating the distal portion of the balloon, a proximal portion of the balloon remains uninflated and a central portion of the balloon remains uninflated.

20. A method of implanting a prosthetic heart valve, the method comprising:
advancing a delivery device to a native aortic valve of a patient while the prosthetic heart valve is crimped over a deflated balloon of the delivery device, the balloon including at least one fold at a proximal end portion of the balloon;
inflating the balloon so that the prosthetic heart valve expands, the prosthetic heart valve advancing distally relative to the native aortic valve while the at least one fold at the proximal end portion of the balloon unfolds during the inflating; and
continuing to inflate the balloon until the prosthetic heart valve expands into contact with the native valve annulus.
